(19) United States
(12) Patent Application Publication
(10) Pub. No.: US 2003/0100819 A1
(43) Pub. Date: May 29, 2003

(54) Hand-held chemical sensing instrument

(75) Inventors: Richard W. Newman, Auburn, NY (US); Dominick Danna, Syracuse, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc.

(21) Appl. No.: 09/995,390

(22) Filed: Nov. 28, 2001

(51) Int. Cl. A61B 5/00

(52) U.S. Cl. 600/300

(57) ABSTRACT

A hand-held medical or industrial diagnostic instrument includes at least one chemical sensing element capable of detecting at least one fluid emitted by a disease and producing an electrical or optical change when said at least one fluid is detected. The instrument further includes a processor having resident circuitry for processing the electrical change generated by said at least one chemical sensing element into a resulting output signal indicative of the disease(s). The at least one chemical sensing element is preferably supported in substantial direct proximity with a target area to be tested.

### FIELD OF THE INVENTION

The invention relates to the field of diagnostic instruments, and more particularly to a hand-held diagnostic instrument having at least one electronic chemical sensing element which can be used for medical and/or industrial purposes.

### BACKGROUND OF THE INVENTION

Chemical sensing devices are repletely well known for detecting the presence of certain vapors, such as carbon monoxide and/or carbon dioxide, in either an industrial or home environment. Likewise, further applications of chemical sensing technology are found in the food processing industry.

To date, however, there are very few known devices which utilize chemical sensors for medical applications.

In addition, most known chemical sensing devices, including each of those referred to above, typically require a housing which retains at least one chemical sensor in a testing chamber. These devices further include means, such as a pump or other similar mechanism, for inputting a portion of the atmosphere of a target area into the housing for evaluation by the chemical sensor(s). An example of such a device is described at length in U.S. Pat. No. 6,234,006 to Sunshine et al. The testing chamber is internally disposed relative to a device housing. Another example found commercially is the Breath Alert Breath Checker manufactured by the Tanika Corporation. Such devices are also described, for example, in U.S. Pat. No. 5,799,102 to Leong which determines the authenticity of a bank note, and U.S. Pat. No. 5,675,070 to Gelperin in which an array of gas sensors are disposed in an interior testing chamber. The gas sensors of the array detect predetermined levels of specified components in a fluid mixture and then produce a sensor pattern which can subsequently be analyzed.

### SUMMARY OF THE INVENTION

It is a primary object of the present invention to overcome the above-noted deficiencies in the prior art.

It is another primary object of the present invention to provide a compact diagnostic instrument which is capable of detecting a hazardous condition in a target environment.

It is yet another primary object of the present invention to provide at least one hand-held medical diagnostic instrument which is capable of detecting the presence of a fluid indicative of a disease or a disease process.

Therefore and according to a preferred aspect of the present invention, there is provided a hand-held medical diagnostic instrument comprising:

at least one chemical sensing element capable of detecting at least one fluid emitted by a disease and producing an electrical or an optical change when said at least one fluid is detected;

processing means for processing said electrical or optical change generated by said at least one chemical sensing element into a resulting signal indicative of said disease; and

supporting means for supporting said at least one chemical sensing element in substantial proximity with a target area to be tested.

The instrument can be any medical diagnostic instrument; for example, a skin surface microscope, an otoscope, an ophthalmoscope, or other similar device, for example, a vaginal scope. According to a preferred embodiment, the instrument includes an instrument head having means for defining a substantially enclosed volume which encompasses at least one chemical sensing element and a medical target area such as an in vivo portion of skin, the eye, nose, vagina, anus, throat and/or other similar area of a patient.

The instrument can also be an endoscope, such as a colonoscope, or a borescope with the target area being an anatomical body cavity or an industrial target such as a chemical fire or pressure vessel, among others.

A preferred form of medical diagnosis can be performed by using miniature chemical sensing elements in conjunction with a hand-held medical diagnostic instrument for detecting the presence of at least one fluid which is indicative of a specified disease of interest. For example, at least one chemical sensing element can be disposed on the blade of a laryngoscope in order to detect the presence of strep throat without requiring a culture from a patient as is required by known apparatus in the field. Similarly, the presence of ketones or acetone as detected by at least one chemical sensing element of the herein described instrument could provide a preliminary indication of diabetes. Moreover, the instrument can include processing means for determining the relative concentration of at least one specific fluid present in a target environment. In addition, a display or other indicating means can be provided to indicate the presence of a disease or other hazardous condition.

The supporting means, for example, can include at least one substrate upon which the at least one chemical sensing element or element array can be disposed. Preferably, an array of chemical sensing elements are disposed upon the supporting substrate which can be positioned in substantial direct contact with the target area. The chemical sensing element arrays and/or the supporting means can also be disposable.

The output of the chemical sensing elements is an electrical or an optical signal when a specific fluid is detected. The generated electrical signal can be transmitted using conventional conductive carriers to a local processing device which provides a comparison between the transmitted signal and a signature indicative of a disease. Alternately, the signal generated by the chemical sensing element(s) can be communicated using IR, RF, or other wireless techniques to a remote diagnostic center or database containing additional processing capability.

According to another preferred aspect of the invention, there is provided a method for detecting the presence of a disease or disease process, said method including the steps of: incorporating at least one chemical sensing element in conjunction with a medical diagnostic instrument, said chemical sensing element being capable of producing an electrical or optical signal when a fluid indicative of said disease or disease process is detected; and disposing said medical diagnostic instrument and said chemical sensing element in substantial direct proximity with a medical target.

Preferably, the method includes the steps of incorporating at least one chemical sensing element into at least one of a hysteroscope and a colposcope and positioning the at least one chemical sensing element in substantial direct proximity with the cervix of a patient for determining the presence of at least one of endometreosis and cancer.

Alternately, the method includes the steps of incorporating at least one chemical sensing element capable of detecting the presence of at least one fluid indicative of a disease in a colonoscope, and positioning said colonoscope in substantial direct proximity with the colon for detecting the presence of at least one of ulcerative colitis and cancer.

An advantage of the present invention is that the presence of a disease, for example, such as strep throat or diabetes, can be determined non-invasively and more expeditiously than by any known means.

A further advantage of the present invention is that the instrument can detect the presence of the hazardous condition without requiring the use of pumps or other apparatus given that the sensor is in substantially direct contact with the target area.

Still another advantage is that the supporting means and/or the chemical sensing element can be disposable and therefore available for single patient use.

These and other objects, features, and advantages will be readily apparent from the following Detailed Description which should be read in conjunction with the accompanying drawings.

### BRIEF DESCRIPTION OF THE DRAWINGS

FIG. 1 is a front elevational view of a hand-held medical diagnostic instrument made in accordance with a first embodiment of the present invention;

FIG. 2 is a side elevational view of the instrument depicted in FIG. 1;

FIG. 3 is a side view, partly in section, of a medical diagnostic instrument made in accordance with a second embodiment of the invention;

FIG. 4 is a side view, partly in section, of a medical diagnostic instrument made in accordance with a third embodiment of the present invention;

FIGS. 5(a), (b) are frontal and side elevational views of a medical diagnostic instrument made in accordance with a fourth embodiment of the present invention;

FIG. 5(c) is a top view of a laryngeal blade assembly of the medical diagnostic instrument depicted in FIGS. 5(a) and (b);

FIG. 6 is a side view of a medical diagnostic instrument made in accordance with a fifth embodiment of the present invention;

FIG. 7 is a side view of a diagnostic instrument made in accordance with a sixth embodiment of the present invention; and

FIG. 8 is a schematic view of a hand-held medical instrument according to the present invention as used in connection with a hospital bed.

### DETAILED DESCRIPTION

The following discussion relates to several embodiments of medical diagnostic instruments incorporating an electronic sensing apparatus. Throughout the discussion which follows, several terms such as “upper”, “lower”, “top”, “bottom” and “lateral”, among others, are used to provide a frame of reference with regard to the accompanying drawings. These terms, however, should not be interpreted as limiting of the inventive concepts described herein. Furthermore, it will also be readily apparent that the herein described concepts are equally applicable outside of the medical field. That is, similar instruments can be created including the concepts herein described which are useful for industrial uses, such as determining the nature of certain chemical fires, determining the presence of certain volatile compounds in a building or structure, and/or a myriad of other potential applications. In addition and for definitional purposes, “fluid” is intended herein to mean any liquid, gas, or vapor. In addition, the term “disease” is also used repletely throughout the discussion. By “disease”, it is intended to cover both diseases which are found in a particular medical target area, such as conjunctivitis in the eye, otitis in the ear, strep throat in the ear and throat, etc. The term is also intended to cover the detection of fluids, for example, the detection of ketones which are indicative of the presence of a systemic disease or disease process.

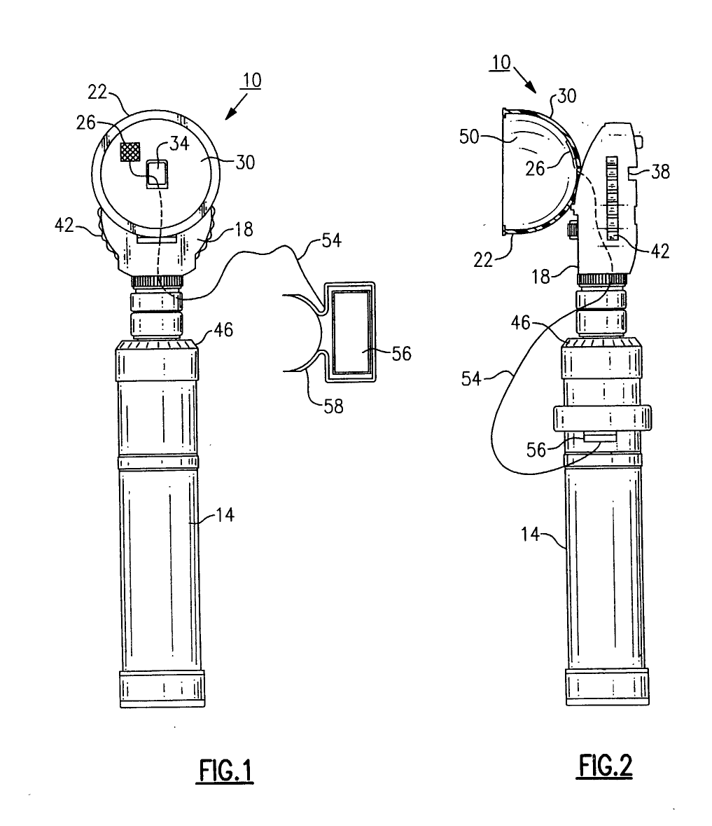

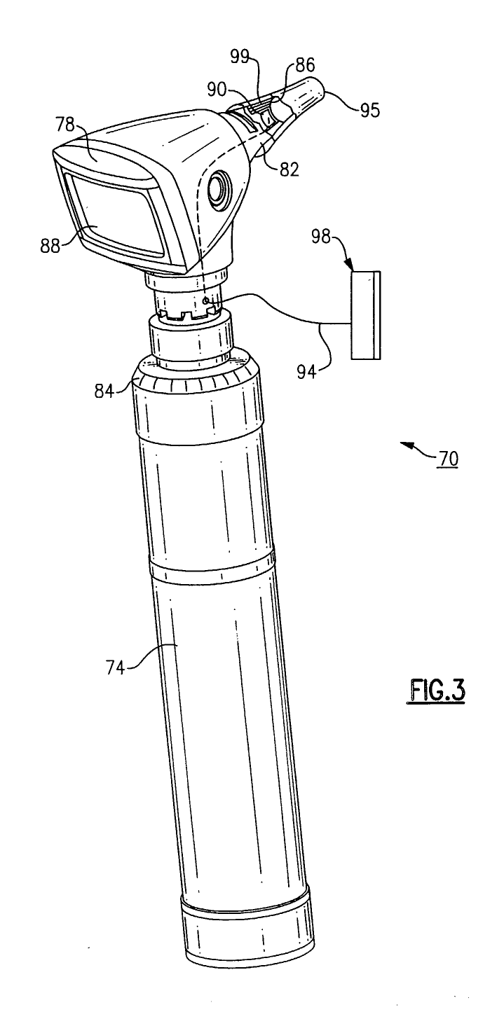

Referring to FIGS. 1 and 2, there is shown a hand-held medical diagnostic instrument made in accordance with a first embodiment of the present invention. The instrument according to this embodiment is an ophthalmoscope 10 used for examining the eyes of a patient which includes a cylindrical handle 14 which can be gripped by the hand of a user as well as an instrument head 18 which is attached to the top of the handle.

The instrument head 18 of the herein described ophthalmoscope 10 includes a horizontal viewing passage therethrough with an aperture 34 on its front or distal side that is brought close to a patient's eye (not shown) and a smaller aperture 38 on its rear or proximal side into which the physician looks. A scalloped edge of a lens wheel 42 is accessible on opposite lateral sides of the instrument head 18 which permits registry with the aperture 38. The lens wheel 42 contains a plurality of lenses (not shown) arranged circumferentially, as is conventionally known, each lens having a different focal length. A light source (not shown), such as a halogen or other miniature bulb, is provided in the body of the cylindrical handle 18 as well as a power source, such as batteries for powering same. The output of the light source is preferably adjusted by a rheostat 46 which is provided on a neck portion of the handle 14. Additional details relating to the standard features and operation of the ophthalmoscope 10 are provided, for example, in U.S. Pat. Nos. 3,978,850, 4,643,546, and 4,998,818, the entire contents of which are herein incorporated by reference.

A semi-spherical enclosure 22 which can be attached by known means to the distal side of the instrument head 18 (or integrally provided therewith) retains an array 26 of chemical sensing elements that are attached by conventional means by epoxy, glass frit, adhesive, or other means. Alternately, the array 26 can also be detachably mounted such as through the use of hook and loop fasteners (not shown) or other commonly known means. The sensing elements of the array 26 are attached to a supporting substrate which is attached along an interior surface of the enclosure 22. The chemical sensing element array 26 according to this embodiment consists of a plurality of miniature polymer gas sensors such as those manufactured by Cyrano Sciences, Inc of Pasadena, Calif., or as described in U.S. Pat. No. 5,571,401 to Lewis et al., U.S. Pat. No. 5,882,497 to Persaud et al., U.S. Pat. No. 6,033,601 to Persuad et al., U.S. Pat. No. 6,093,308 to Lewis, and U.S. Pat. No. 6,013,229 to Lewis, the entire contents of each are herein incorporated by reference. It will be readily apparent, however, that other known chemical sensing elements such as organic gas sensors, conductive composites, metal oxide field effect transistors, surface acoustic wave (SAW) sensors, piezoelectric sensors, metal oxide sensors, and others, can easily be substituted without significantly deviating from the inventive concepts of the invention. Each of the chemical sensing elements of the array 26 are capable of detecting a specific fluid (e.g., gas, liquid, or vapor), the presence of which produces an electrical change. Chemical reactions on the sensors can also cause changes which can be subsequently detected by optical means. These miniature optical chemical sensors, for example, those manufactured by Illumina, Inc., of San Diego Calif., are also within the ambit of the present invention.

Due to the overall sensitivity of the miniature polymer gas sensors of the chemical sensing element array 26 utilized in this embodiment, at least one temperature sensor and/or humidity sensor and/or pH sensor (not shown) are also preferably attached in a conventional manner either to the interior surface of the semi-spherical enclosure 22 or otherwise in the immediate vicinity of the chemical sensing element array 26. The semi-spherical enclosure 22, when brought into direct, intimate contact with the patient, defines a substantially sealed and enclosed volume or head space 50 which in effect creates a test chamber, the chamber including the chemical sensing element array 26 and the target atmosphere. It should be further noted that though enclosure 22 is substantially semi-spherical, other suitable shapes or geometries capable of defining a head space can be utilized. As further described below, the semi-spherical enclosure can also be used to produce a head space for interrogating a number of different anatomical target areas, including body cavities such as the vagina, nose, throat, anus or an in vivo potion of skin.

In operation, the ophthalmoscope 10 is used in the conventional way to examine the eye using the lens wheel 42 and contained light source. The semi-spherical enclosure 22 includes a rear opening to allow optical access to the distal side aperture 34, the enclosure further providing a substantially sealed area about the eye (not shown) as previously noted. As noted above, and the presence of certain analytes (fluids), for example, those which might be representative of conjunctivis or other disease, certain of the chemical sensing elements of the array 26 will produce a change in an electrical or optical characteristic (such as resistance, transconductance, conductance, impedance, resonant frequency, voltage, inductance, capacitance, or other perceivable electrical or optical parameter), the sensing elements of the array each being connected through traces (not shown) provided along the supporting substrate to corresponding electrical conductors 54 to provide an electrical path to an attached microprocessor 56 or alternately to a display (not shown).

The microprocessor 56 includes suitable memory as well as processing circuitry which analyzes the electrical signals generated by the chemical sensing element array 26, the herein described microprocessor including a snap-on clip 58 to attach same to the cylindrical handle 14 of the ophthalmoscope 10. Preferably, the microprocessor 56 includes certain integrated processing electronics including an analog-digital (A/D) converter as well as resident control and timing circuitry which is used in conjunction with a reference crystal in order to detect the amount of electrical change by each of the sensing elements of the array 26 for processing, such as comparing to a stored look-up table and then outputting the results to an LCD or other suitable display. The microprocessor 56 can further include a set of internal lithium batteries (not shown) for powering the processor and the chemical sensing element array 26, or each can be powered by the batteries (not shown) which are already contained within the instrument handle 14. Each of the above features are described in greater detail in commonly owned U.S. Ser. No. 09/663,698 to Newman et al., the entire contents of which are herein incorporated by reference.

The microprocessor 56 preferably includes sufficient memory for storing the values of the electrical signals generated by the chemical sensing element array 26. According to an alternate embodiment, the instrument can include a transmitting antenna and receiver. These features are described in greater detail in U.S. Ser. No. 09/884,224, which is also herein incorporated by reference in its entirety. In this manner, a remotely generated signal can cause wireless (RF, IR, etc.,) transmission of the electrical signal values stored by the memory of the microprocessor 56 for downloading to a local and/or remote processing unit (not shown) having greater processing capability.

Referring to FIG. 3, there is shown a diagnostic instrument made in accordance with a second embodiment of the present invention. In this instance, the diagnostic instrument is an otoscope 70 which is used for examining the outer ear. Like the preceding, the present instrument includes a hand-grippable instrument handle 74 as well as an instrument head 78 which is attached in a known manner to the top of the instrument handle. The instrument head 78 of the herein-described otoscope 70 includes a frustoconical insertion or speculum portion 82 which is sized to be fitted a predetermined distance into the ear canal (not shown) of a patient and further includes a distal or front tip opening 86 and may include a tip 95 enclosing the ear canal. A magnifying eyepiece 88 is provided on the opposite rear or proximal side of the instrument head 78, the head possibly further including an enclosed optical system (not shown), such as an objective or relay lens assembly, used to transmit light reflected from the ear through the distal tip opening 86 and to the eyepiece 88. As in the preceding, a halogen or other miniature bulb (not shown) contained within the instrument handle 74 is used to illuminate the target. Preferably, the output of the light source is preferably controlled by a rheostat 84 provided on the upper portion of the instrument handle 74. The illumination output is directed, for example, using a series of optical transmitting fibers 99 that extend into the interior of the insertion portion 82 and encircle the distal tip opening 86. Alternately, white LEDs (not shown) or other light sources can be utilized, the choice of light source not being an essential part of the present invention. In each instance, power is supplied to the light source through batteries (not shown) which are retained within the handle 74 of the instrument 70. The standard features of the herein described otoscope 70 and operation thereof can be found, for example in U.S. Pat. Nos. 3,992,158 and 5,919,130, the entire contents of each herein being incorporated by reference.

For purposes of this invention and still referring to FIG. 3, a chemical sensing element array 90 is attached, detachably or otherwise, to an interior surface of the frusto-conical insertion portion 82 or the tip 95, preferably adjacent the distal tip opening 86. As in the preceding, the chemical sensing element array 90 according to this embodiment are miniature polymer gas sensors manufactured by Cyrano Sciences, Inc of Pasadena, Calif., such as those described in previously incorporated U.S. Pat. Nos. 5,571,401, 5,882,497, 6,033,601, 6,093,308, and 6,013,229. As previously noted, however, it will be readily apparent that other known chemical sensing elements, such as organic gas sensors, conductive composites, metal oxide field effect transistors, surface acoustic wave sensors, piezoelectric sensors, metal oxide sensors, optical chemical sensors and others, can easily be substituted without significantly deviating from the inventive concepts of the invention.

The chemical sensing element array 90 is preferably disposed on a supporting substrate, such as that described in previously incorporated U.S. Ser. Nos. 09/663,698 and 09/884,224, wherein a series of traces (not shown in FIG. 3) which extend from each of the individual chemical sensing elements through the interior of the insertion portion 82 and the handle 74 along corresponding electrical conductors 94 to a microprocessor 98 which can be connected to the instrument handle 74, such as using the exemplary clip-like attachment previously shown in FIGS. 1 and 2. The sensing element array 90, as well as the microprocessor 98 which includes the processing circuitry described previously and also as described in incorporated U.S. Ser. No. 09/884,224 can be powered using the batteries (not shown) contained within the handle 74, or alternately through dedicated batteries (not shown) provided with the microprocessor 98.

In use, the otoscope 70 is used in a conventional manner to examine the ear (not shown) of a patient by placing the distal part of the insertion portion 82 into the ear of the patient and by directing light from the contained light source through the distal tip opening 86. The image received through the magnifying eyepiece 88 is then inspected by the physician to detect, for example, the presence of infection. In addition, the chemical sensing element array 90 is also exposed directly to the atmosphere of the outer ear and in the presence of certain analytes, for example, those which might be representative of otitis or other malady including upper respiratory infections such as strep throat. As a result, certain of the chemical sensing elements of the array 90 will produce a change in an electrical and/or optical characteristic, (such as transconductance, conductance, impedance, resonant frequency, voltage, inductance, capacitance, and resistance, etc.,) the sensing elements of the array each being connected through traces (not shown) provided along the supporting substrate to the corresponding electrical conductors 94 to provide an electrical path to the attached microprocessor 98. The microprocessor 98 includes processing circuitry which analyzes the electrical signals generated by the chemical sensing element array 90. As in the preceding, a transmitter (not shown) could permit wired or wireless transmission of stored electrical signal values from the microprocessor 98, such as to a remote processing unit (not shown).

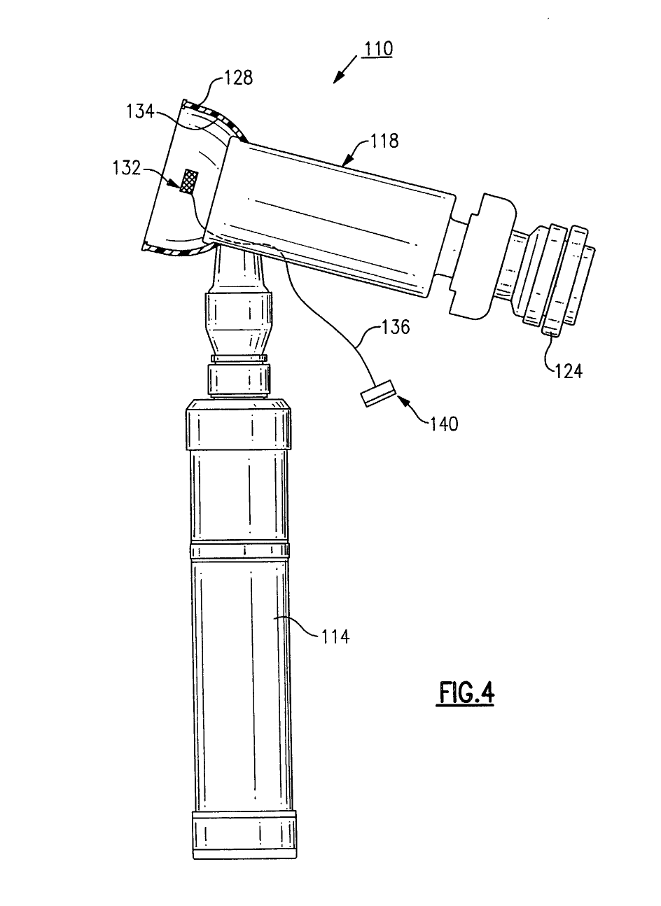

Referring to FIG. 4, there is shown a diagnostic instrument made in accordance with a third embodiment of the present invention. In this instance, the instrument is a skin surface microscope 110 which includes an instrument head 118 attached to a hand-grippable handle 114, the instrument head having a distal side (not shown) and a proximal side which includes an eyepiece 124. The interior of the instrument head 118 includes an optical system which magnifies a corresponding image of a target which is brought to the eyepiece 124. As in the preceding, a light source (not shown) and batteries (not shown) are contained within the instrument handle 114 for illuminating a target of interest, in this instance, an in vivo section of skin. Details relating to the specific features of a skin surface microscope are provided, for example, in previously incorporated U.S. Pat. No. 3,978,850.

Still referring to FIG. 4, a semi-spherical enclosure 128 is attached to the distal side of the instrument head 118, the enclosure including a chemical sensing element array 132, similar to those previously described above. The sensing element array 132 is attached such as by glass frit, epoxy, or other means to a supporting substrate which is attached fixedly or detachably by conventional means to an interior surface 134 of the enclosure 128. The sensing element array 132 includes a plurality of polymeric, organic or other fluid sensing elements, such as those described above, each of the sensing elements including traces provided on the substrate which permit electrical or optical signals to be carried along electrical or optical conductors 136 to an attached microprocessor 140 having suitable memory and processing circuitry for processing the generated electrical signals, as described below, and in incorporated U.S. Ser. No. 09/884,224.

In operation, the presently described skin surface microscope 110 is used in its intended conventional manner by placing the semi-spherical enclosure 128 over and in direct intimate contact with a target of interest and viewing same as illuminated by the contained light source (not shown) through the eyepiece 124 of the instrument head 118 to examine an in vivo portion of skin and to detect the occurrence of moles, lesions, warts, skin cancers, psoriasis, allergic reactions such as rashes, and the like. In addition, and without detracting from this original use, the chemical sensing element array 132 can be used to detect the presence of at least one fluid in the vicinity of the in vivo skin portion (not shown) which is indicative of a disease, such as diabetes. As in the preceding, the detection of a specific vapor produces an electrical or optical change in at least one chemical sensing element, this change(s) being transmitted to the microprocessor 140 having suitable memory for storing the signal value.

According to the present embodiment, the microprocessor 140 contains sufficient processing circuitry and a look up table to compare the sensor pattern of the array 132 in order to indicate the presence of a disease process. Alternately and as described above, the stored electrical signal values could be transmitted by known wire or wireless (IR, RF) technologies to a local or remote processing unit (not shown). The instrument 110 can further include an LCD (not shown) or other means to indicate a result.

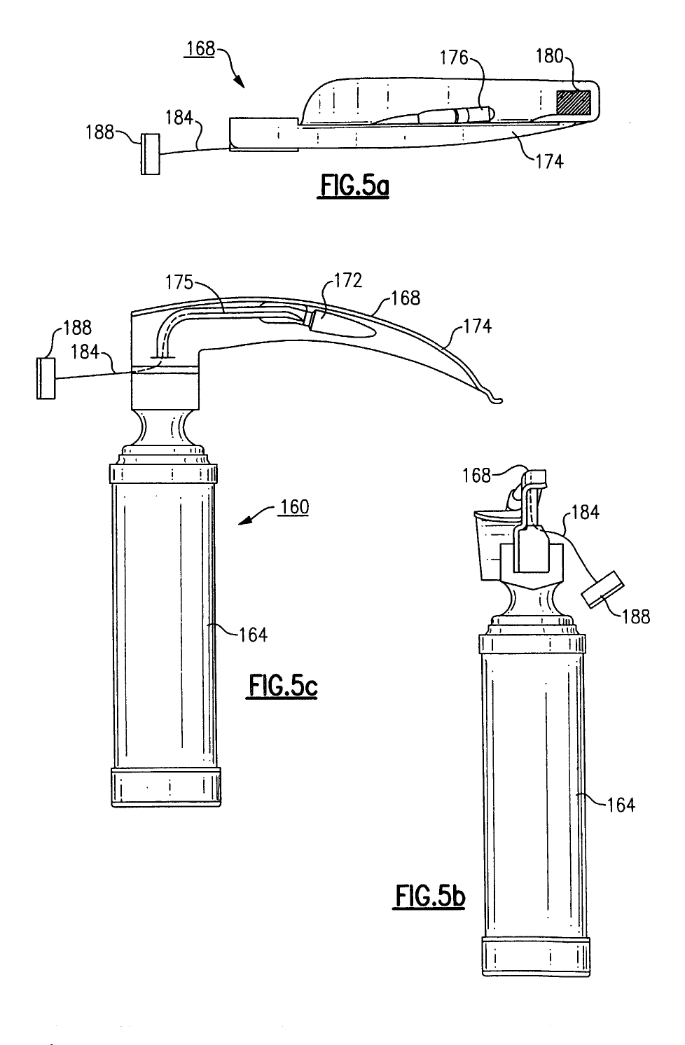

Referring to FIGS. 5(a)-5(c), there is shown a medical diagnostic instrument made in accordance with a fourth embodiment of the present invention. In this instance, the instrument is a laryngoscope 160 which is generally used to displace the tongue and to inspect the throat/larynx of a patient preferably before introducing tracheal tubes and the like for intubation. The laryngoscope 160 includes a hand-grippable cylindrical handle 164, similar to that previously described, as well as a releasable blade assembly 168 which is attached to the top of the handle. An exemplary blade assembly is described in commonly assigned U.S. Pat. Nos. 6,013,026 and 6,213,927, the entire contents of each are herein incorporated by reference.

According to this embodiment, the laryngeal blade assembly 168 includes a laryngeal blade 174, sometimes referred to as a spatula, which is an elongated member made from a biocompatible material, such as stainless steel, which is sized to examine the larnyx. A tube with wires 175 extends from the handle 164 terminating at a halogen bulb 176 which extends from a distal opening in a receiving pocket 172. This particular design of instrument is not critical to the present invention; that is, other laryngoscopes include a halogen bulb disposed within the handle and a plurality of optical fibers extending into the hollow light pipe 175 in order to transmit the light from the bulb to the distal tip of the laryngeal blade 174.

Still referring to FIGS. 5(a)-5(c), an array 180 of chemical sensing elements, such as the polymeric gas sensing elements which have been previously described above, is attached by known means to a supporting substrate which is mounted to a distal end of the laryngeal blade 172.

Like the previous embodiments, the array 180 of chemical sensing elements includes a set of traces provided on the supporting substrate which are connected to electrical conductors 184 extending to a microprocessor 188. As in the preceding, the microprocessor 188 contains suitable memory for storing electrical signal values generated by the array as well as processing circuitry, such as described in previously incorporated U.S. Ser. No. 09/884,224, for analyzing the generated signals.

As in each of the preceding examples, the herein described instrument 160 can be used in the conventional manner to inspect the larynx, the distal end of the blade 172 extending into the throat of a patient permitting, for example, the detection of certain fluids given off which are indicative of upper respiratory infections such as strep throat or other fluids such as ketones or acetone indicative of diabetes, or cancer.

Alternately an in lieu of a laryngoscope blade, a tongue blade or depressor can be used to similarly interrogate the throat of a patient. Such a tongue depressor design is described in copending U.S. Ser. No. 09/884,224, previously incorporated, in its entirety.

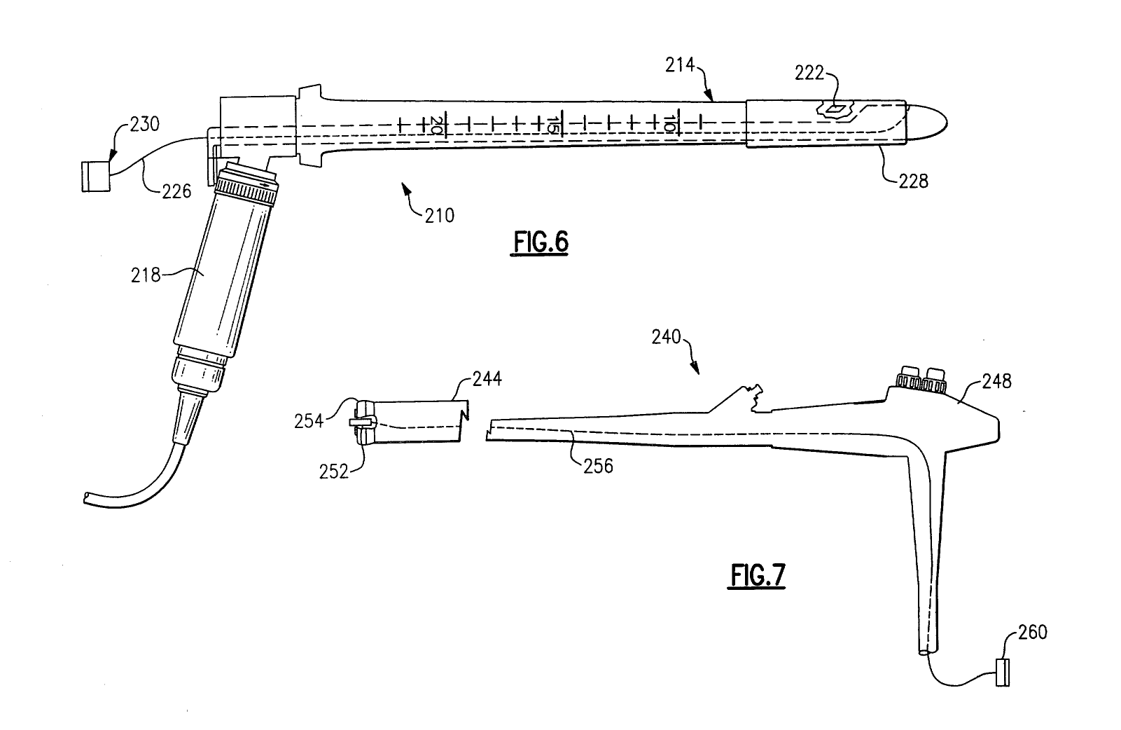

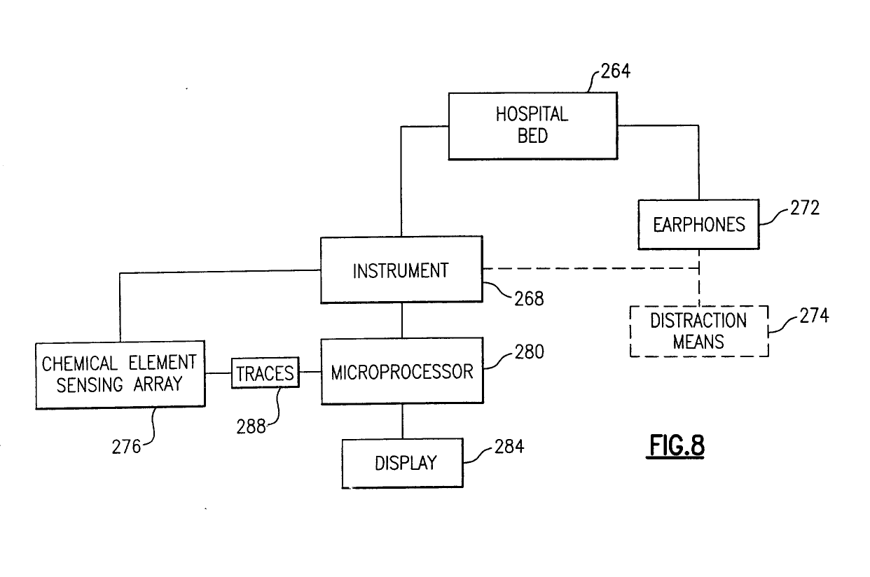

Referring to FIGS. 6 and 7, medical diagnostic instruments are depicted which are made in accordance with respective fifth and sixth embodiments of the present invention, each incorporating at least one chemical (fluid) sensing element. In these examples, the instruments are a medical endoscope, such as a rigid sigmoidoscope 210, shown in FIG. 6, which is used to inspect the sigmoid-colon and a flexible endoscope 240, such as a colonoscope that is shown in FIG. 7.

Referring to FIG. 6, the sigmoidoscope 210 includes an elongated insertion portion 214 which is connected to a handle section 218 such as described in, for example, U.S. Pat. No. 4,353,358 to Emerson et al., among others. The insertion portion 214 is a cylindrical stainless steel or plastic tube which is shaped and sized for fitting into the rectum and including therein an illumination system which permits visualization by the physician either through an eyepiece or through video endoscopy. Details relating to the general operation and features of the sigmoidoscope 210 are found in the above U.S. Pat. No. 4,353,358 which is herein incorporated by reference in its entirety.

According to the present embodiment, a chemical sensing element array 222 is attached by known means to a distal end 228 of the insertion portion 214. As in the preceding, the array includes a plurality of polymeric, organic, or other type of miniature fluid sensing elements which in the presence of certain analytes will change an electrical characteristic. Examples of such sensors are provided in the preceding embodiments. The sensing elements are provided on a supporting substrate (not shown) made from a biocompatible material and including a set of traces which are connected to a set of electrical conductors 226 extending to a microprocessor 230. The microprocessor 230 contains memory and processing circuitry as described in each of the preceding embodiments and as further described in previously incorporated U.S. Ser. No. 09/884,224. Likewise, wired or wireless interconnection with a remote or local processing unit having additional capability is also contemplated, though not shown in FIG. 6.

As in the preceding, the instrument 210 can be used in its intended manner by the physician for examination purposes, with additional capability being provided by the attached array 222 which can be used to detect the specific fluids present in the examined environment, indicative of disease, for example, ulcerative colitis, diverticulitis, cancer, and ilitis among others.

Referring to FIG. 7, a flexible endoscope or borescope 240 is shown which is constructed similarly to the endoscope of FIG. 6; that is, including an insertion portion 244 and a control section 248. A chemical sensing element array 252, similar to that shown in FIG. 6, is attached by conventional means to a distal end 254 of the insertion portion 244, the array being electrically connected through a series of conductors 256 to a microprocessor 260. The instrument shown is a colonoscope though it should be readily apparent that other medical endoscopes, such as the group consisting of laparoscopes, laryngoscopes, hysteroscopes, gastroscopes, otoscopes, laparoscopes, colposcopes, arthoscopes, vaginal scopes, esosphogoscopes, anoscopes, angioscopes, thoraxoscopes, and cystoscopes can also be utilized.

A number of different medical diagnosis procedures can be performed using certain of the above noted instruments. For example, at least one of ulcerative colitis and cancer can be diagnosed using at least one chemical sensing element or elements capable of detecting the presence of at least one vapor indicative of a disease and mounting the element(s) in the manner described above in a colonoscope.

Similarly, the presence of endometreosis or cancer or pre-cancer in the cervix can also be detected by incorporating at least one chemical sensing element or an array of such elements, such as those described herein, in conjunction with at least one of a hysteroscope and a colposcope and positioning the chemical sensing element(s) in substantial direct proximity with the cervix of a patient. The herein described endoscope can further include an isolating enclosure similar to 22 to allow build-up of concentration of fluids in the target environment.

As noted, industrial borescopes include designs similar to that illustrated in FIG. 7 which can be used for inspection purposes. It is contemplated that this apparatus can also be configured to include sensing arrays such as described herein which can be added to permit detection of specific vapors or other fluids.

Referring to FIG. 8, there is shown a schematic view to better describe the overall versatility of the above invention.

In this instance, a hand-held medical instrument (a sphygmomanometer 268 used to measure mean arterial blood pressure) includes a chemical sensing element array 276 which is releasably or fixedly attached to the interior side of an inflatable blood pressure sleeve. The instrument 268, including the chemical sensing element array 276, a microprocessor 280 and a display 284 is attached, either directly or in close proximity to a hospital bed, shown schematically as 264.

The chemical sensing element array 276 includes at least one chemical sensing element of detecting a specific fluid through an electrical or optical change. This change is transmitted through traces 288 to the microprocessor 280 which contains appropriate processing circuitry. If the specific fluid, indicative of a disease or disease process is detected, then an appropriate signal is sent to the display 284.

According to this embodiment, a set of earphones 272 are provided. These earphones 272 can be used to distract the patient during measurement. Alternate distracting means, shown in phantom as 274, can also be contemplated, for example, an audio, video or other distraction can be provided. In addition, the earphones 272 cover the ears and thereby can produce an enclosed headspace in a similar manner to the semi-spherical enclosed volume 50, FIG. 2, for use with a device (not shown) such as previously described for determining the presence of fluid indicative of a disease within the ear. However, because the ear volume is relatively small, it may take considerable time to build up a measurable concentration of fluid within the headspace. Therefore, the earphones 272 can be also be used to distract the patient (not shown) during measurements.

It should also be apparent that the instrument could wirelessly transmit the electrical or optical change/signal to a remote processor unit (not shown).

### PARTS LIST FOR FIGS. 1-8

10 ophthalmoscope

14 handle

18 instrument head

22 enclosure

26 chemical sensing element array

30 interior surface

34 aperture

38 aperture

42 lens wheel

46 rheostat

50 enclosed volume

54 conductors

56 microprocessor

58 clip

70 otoscope

74 handle

78 instrument head

82 frusto-conical insertion portion

84 rheostat

86 distal tip opening

88 magnifying eyepiece

90 chemical sensing element array

94 electrical conductors

95 tip

98 microprocessor

99 optically transmitting fibers

110 skin surface microscope

114 handle

118 instrument head

124 eyepiece

128 enclosure

132 chemical sensing element array

134 interior surface

136 conductors

140 microprocessor

160 laryngoscope

164 handle

168 laryngeal blade assembly

172 receiving pocket

174 blade

175 tube with wires

176 bulb

180 chemical sensing element array

184 electrical conductors

188 microprocessor

210 sigmoidoscope

214 insertion portion

218 handle portion

222 chemical sensing element array

226 conductors

228 distal end

230 microprocessor

240 endoscope

244 insertion portion

248 control section

252 chemical sensing element array

254 distal end

256 conductors

260 microprocessor

264 hospital bed

268 instrument (sphygmomanometer)

272 earphones

274 distracting means

276 chemical sensing element array

280 microprocessor

284 display

It should be apparent that other variations and modifications can be realized within the intended scope of the present invention in addition to the embodiments described herein.

We claim:

1. A hand-held medical diagnostic instrument comprising:
at least one chemical sensing element capable of detecting at least one fluid emitted by a disease and producing an electrical or optical change when said at least one fluid is detected; and
processing means for processing said electrical change generated by said at least one chemical sensing element into a resulting output signal indicative of said disease; and
supporting means for supporting said at least one chemical sensing element in substantial proximity with an area to be tested.

2. An instrument as recited in claim 1, including indicating means for indicating when said at least one fluid is detected.

3. An instrument as recited in claim 2, wherein said indicating means is a display.

4. An instrument as recited in claim 1, wherein said instrument is a skin surface microscope.

5. An instrument as recited in claim 4, including means for creating a substantially enclosed volume encompassing at least one chemical sensing element and an in vivo portion of skin serving as a target area.

6. An instrument as recited in claim 5, wherein said instrument can interrogate a medical target area selected from the group consisting of at least one of the ear, mouth, eyes, vagina, anus, urinary tract, nose, throat and an invivo portion of skin using said means for creating a substantially enclosed volume.

7. An instrument as recited in claim 1, wherein said instrument is an ophthalmoscope.

8. An instrument as recited in claim 7, wherein said at least one chemical sensing element is capable of detecting conjunctivitis.

9. An instrument as recited in claim 7, including means for creating a substantially enclosed volume encompassing at least one chemical sensing element and an eye of a patient.

10. An instrument as recited in claim 1, wherein said instrument is an endoscope and said area of interest is a body cavity.

11. An instrument as recited in claim 10, wherein said instrument is selected from the group consisting essentially of colonoscopes, laryngoscopes, hysteroscopes, gastroscopes, otoscopes, laparoscopes, colposcopes, arthoscopes, vaginal scopes, esosphogoscopes, anoscopes, angioscopes, thoraxoscopes, and cystoscopes.

12. An instrument as recited in claim 1, wherein said instrument is capable of detecting the presence of strep throat in a patient without requiring a culture therefrom.

13. An instrument as recited in claim 1, wherein said supporting means includes a substrate upon which said at least one chemical sensing element is disposed, said substrate being sized to be positioned in the mouth or throat of a patient, wherein said at least one chemical sensor is capable of detecting the presence of diabetes.

14. An instrument as recited in claim 13, wherein said at least one chemical sensing element is capable of detecting the presence of at least one of ketones and acetone, said processing means being capable of calculating the concentration thereof as detected by said at least one chemical sensing element.

15. An instrument as recited in claim 14, including indicating means for indicating the concentration of said at least one of ketones and acetone.

16. An instrument as recited in claim 1, including indicating means for indicating the concentration of said at least one of ketones and acetone.

17. An instrument as recited in claim 15, wherein said indicating means includes a display connected to said processing means.

18. An instrument as recited in claim 1, wherein said indicating means includes a display connected to said processing means.

19. An instrument as recited in claim 1, wherein said instrument is a sphygmomanometer including a cuff mounted onto the limb of a patient and in which said at least one chemical sensing element is mounted on said cuff such that chemicals emitted by and through the skin of a patient can be detected.

20. An instrument as recited in claim 1, including means for installing said instrument in at least close proximity to a hospital bed.

21. An instrument as recited in claim 20, wherein said instrument is capable of interrogating a medical target from the group consisting of at least on of the ear, mouth, eyes, vagina, anus, urinary tract, nose, throat, and an invivo portion of skin.

22. An instrument as recited in claim 1, wherein said instrument is a sphygmomanometer.

23. An instrument as recited in claim 1, including distracting means for distracting a patient when the device is being used.

24. An instrument as recited in claim 23, wherein said distracting means includes a set of earphones.

25. An instrument as recited in claim 1, wherein said instrument is an industrial borescope.

26. An instrument as recited in claim 1, wherein at least one of said supporting means and said at least one chemical sensing element is disposable.

27. An instrument as recited in claim 1, including means for transmitting signals from said at least one chemical sensing element to a remote processing unit.

28. An instrument as recited in claim 27, wherein said transmitting means includes a wireless link between said at least one chemical sensing element and said remote processing unit.

29. An instrument as recited in claim 1, wherein said instrument is battery powered.

30. A method for detecting the presence of a disease or disease process, said method including the steps of:
incorporating at least one chemical sensing element in conjunction with a medical diagnostic instrument, said chemical sensing element being capable of producing an electrical or optical signal when a fluid indicative of said disease or disease process is detected; and
disposing said medical diagnostic instrument and said chemical sensing element in substantial direct proximity with a medical target.

31. A method as recited in claim 30, including the additional steps of:
incorporating at least one chemical sensing element in conjunction with at least one of a hysteroscope and a colposcope; and
positioning said at least one chemical sensing element in substantial direct proximity with the cervix of a patient, for detecting the presence of at least one of endometreosis and cancer.

32. A method as recited in claim 30, comprising the additional steps of:
incorporating at least one chemical sensing element capable of detecting the presence of at least one fluid indicative of a disease in at least one of a colonoscope, a gastroscope and a laparoscope; and
positioning said at least one chemical sensing element in substantial direct contact with the sigmoid-colon of a patient for detecting the presence of at least one of ulcerative colitis and cancer.

33. A method as recited in claim 30, including the additional steps of:
disposing at least one chemical sensing element onto at least one of a laryngoscope blade and a tongue depressor; and
positioning said one of said laryngeal blade and tongue depressor including said at least one chemical sensing element within the throat of a patient for detecting the presence of at least one of diabetes, upper respiratory infections and cancer.

34. A method as recited in claim 30, including the additional steps of:
disposing at least one chemical sensing element in conjunction with an otoscope; and
positioning said at least one chemical sensing element into the ear of a patient for detecting the presence of at least one of otitis and upper respiratory infections.
